(19) United States
(12) Patent Application Publication
(10) Pub. No.: US 2007/0275066 A1
(43) Pub. Date: Nov. 29, 2007

(54) Sustained release drug delivery system composed of water insoluble polymer

(75) Inventors: Sun Hang Cho, Yuseong-gu (KR); Byung Cheol Shin, Yuseong-gu (KR); Soon Hong Yuk, Yuseong-gu (KR); Jin A. Seo, Seo-gu (KR); Ji Sun Park, Yangsan City (KR)

Correspondence Address: THE WEBB LAW FIRM, P.C., 700 KOPPERS BUILDING, 436 SEVENTH AVENUE, PITTSBURGH, PA, 15219, US

(73) Assignee: Korea Research Institute of Chemical Technology, Yuseong-gu (KR)

(21) Appl. No.: 11/508,348

(22) Filed: Aug. 23, 2006

(30) Foreign Application Priority Data
May 26, 2006 (KR) 10-2006-0047682

(51) Int. Cl. A61K 31/517; A61K 31/445; A61K 9/24

(52) U.S. Cl. 424/472; 514/252.16; 514/317; 514/649; 514/594; 514/521

(57) ABSTRACT

The present invention relates to a sustained release drug delivery system composed of a water insoluble polymer, and more particularly, to a sustained release drug delivery system comprising: a crystalline core material; an active ingredient layer which is formed on an outer surface of the crystalline core material and comprises a pharmacologically active ingredient and a water insoluble polymer; and a sustained release layer which is formed on an outer surface of the active ingredient layer and comprises a sustained release film forming material. The present invention also relates to a sustained release drug delivery system which releases an effective drug in an aqueous solution or a body fluid for 24 hours by using a sustained release film forming material and a plasticizer together to provide sustained release of a pharmacologically active ingredient.

### TECHNICAL FIELD

The present invention relates to a sustained release drug delivery system composed of a water insoluble polymer, and more particularly, to a sustained release drug delivery system comprising a crystalline core material, an active ingredient layer which is formed on an outer surface of the crystalline core material and comprises a pharmacologically active ingredient and a water insoluble polymer, and a sustained release layer which is formed on an outer surface of the active ingredient layer and comprises a sustained release film forming material. The present invention also relates to a sustained release drug delivery system which releases an effective drug in an aqueous solution or a body fluid for 24 hours by using a sustained release film forming material and a plasticizer together to provide sustained release of a pharmacologically active ingredient.

### BACKGROUND ART

The drug delivery rate into the body should be controlled when a drug is absorbed too slowly and bioavailability of the drug is low or when the drug is absorbed too quickly and is lost out of the body. For this purpose, mechanical systems, osmosis pumps, membranous diffusion controlling systems, systems utilizing polymer materials that are degradable or nondegradable in the body, and so forth, are employed alone or in combination.

Korean Patent Laid-Open Publication No. 2001-23358 discloses a sustained release drug delivery system, which is composed of three different layers. This is a method of cotrolling the rate at which a drug passes through or diffuses into multiple layers, which requires long time in the preparation due to multiple processing steps.

Korean Patent Laid-Open Publication No. 2000-69800 discloses a method of preparing a porous matrix type sustained-release formulation by the emulsion method. In this method, a polymer compound and a surfactant are dissolved in an organic solvent, and then a drug-containing aqueous solution is dispersed in the solution and is stirred to obtain an emulsion. Then, the emulsion is prepared into a desired matrix form and immediately lyophilized. Drying is then performed at room temperature for a certain time, i.e., until the surface of the matrix begins to harden and drying is performed again in vaccum to obtain a porous matrix. This method has disadvantages in uniformity of the membrane, stability of the resultant polymer membrane or enlarged poresize.

U.S. Patent Application Publication No. 2001-0048943 teaches a method of preparing an osmosis system for controlling a drug release, which comprises obtaining granules by mixing venlafaxine, mannitol and povidone with a homogenizer and thoroughly immersing the mixture in alcohol before drying, and performing coating on an outer surface of a venlafaxine pellet of the resulting core. This method is limited in that it takes a long time to prepare granules.

U.S. Patent Application Publication No. 2003-0215507 discloses a method of preparing granules of venlafaxine. The granules are prepared by mixing a drug and at least one binder (hydroxypropyl methyl cellulose, HPMC), damping the mixture with water or a solvent, and then extruding the resultant using a plastic mesh. The resulting granules was dried and coated with a sustained release base (ethyl cellulose, EC). However, the resulting granules have a broad particle distribution since it is impossible to control a particle size in the granulation, as well as the non-uniform particle distribution can lead to inconsistent dissolution rate. When only granules with a uniform size are selected to achieve a consistent dissolution rate, availablility of raw materials is reduced.

Despite the reduction in availablility of raw materials, the plastic mash is used since when coating is performed using a fluidized bed coater, due to a high viscosity of venlafaxine, granules agglomerate or the solvent slowly evaporates, which causes incomplete drying and agglomeration of particles.

Meanwhile, 1-[2-(dimethylamino)-1-(4-methoxyphenyl)ethyl]cyclohexanol, i.e., venlafaxine is a phenethylamine bicyclic antidepressant called as the second generation antidepressant and is used to treat depression and obsessive-compulsive syndrome. This drug selectively inhibits absorption of serotonin, norepinephrine and dopamine to reduce efficacy thereof, does not exhibit anticholinergic, sedative and cardiovascular effects, which are observed in other antidepressants, and does not inhibit the effect of a monoamine oxidase. Venlafaxine has been approved as an antidepressant by FDA and can also be used to treat chronic fatigue syndrome, emotion disorder and obsessive-compulsive syndrome.

Thus, the present invention provides a sustained release drug delivery system having uniform particles and improved sustained-release of a drug by using at least one water-insoluble polymer to reduce the viscosity of the drug and to improve sustained-release of the drug.

### DISCLOSURE OF THE INVENTION

It is an object of the present invention to provide a sustained release drug delivery system comprising: a crystalline core material; an active ingredient layer which is formed on an outer surface of the crystalline core material and comprises a pharmacologically active ingredient and a water insoluble polymer; and a sustained release layer which is formed on an outer surface of the active ingredient layer and comprises a sustained release film forming material.

It is another object of the present invention to provide a sustained release drug delivery system which is composed of a dual coating layer and has a zero-order drug release rate due to slow release of a pharmacologically active ingredient.

To achieve the above objects, the present invention provides a sustained release drug delivery system comprising: a crystalline core material (A); an active ingredient layer (B) which is formed on an outer surface of the crystalline core material and comprises a pharmacologically active ingredient and a water insoluble polymer; and a sustained release layer (C) which is formed on an outer surface of the active ingredient layer and comprises a sustained release film forming material.

Hereinafter, the present invention is described in detail.

The present invention relates to a sustained release drug delivery system. The drug delivery system can controlled-release a pharmacologically effective ingredient at a zero-order rate. The release rate of the pharmacologically effective ingredient can be efficiently controlled by varying the type of the water insoluble polymer, the thickness of the active ingredient layer, the type of the sustained release film formimg material and the thickness of the sustained release layer, thereby improving productivity and simplifying the preparation process.

As used herein, the term “drug delivery system” means a formulation delivering a pharmacologically active ingredient to a subject in need thereof.

As used herein, the term “water insoluble polymer” means a pH-independent polymer which is insoluble in both acid and base.

The sustained release drug delivery system of the present invention comprises: a crystalline core material (A); an active ingredient layer (B) which is formed on an outer surface of the crystalline core material (A) and comprises a pharmacologically active ingredient and a water insoluble polymer; and a sustained release layer (C) which is formed on an outer surface of (B) the active ingredient layer and comprises a sustained release film forming material.

For the crystalline core material (A), a pellet prepared from sugar, sugar granule, nonpareil, sugarsphere and the like as a granular material may be used. The whole granule size may be determined according to the size of the pellet used as the crystalline core material. Preferably, the pellet has a diameter ranging from 300 to 900 μm, and more preferably 600 to 710 μm. If the diameter of the pellet is less than 300 μm, the amount of an excipient used in the coating increases and coating yield is low. If it is greater than 900 μm, the resultant granule may become larger so that it is difficult to fill the granule in a gelatine hard capsule and the granule can be insufficiently filled resulting to cause problems in production. That is, when the crystalline core material is small, a large amount of granules should be admistrated due to low effective content of the pharmacologically active ingredient in an individual granule. Thus, the size of the granule orally administrated with a hard capsule is adequate in the above-described range.

In the sustained release drug delivery system of the present invention, the active ingredient layer (B) is spherically formed on a surface of the crystalline core material and comprises a pharmacologically active ingredient.

The pharmacologically active ingredient means a drug and equivalent thereof and also includes physiologically or pharmacologically active ingredients produced from the drug in the body. At least one pharmacological active ingredient can be comprised in the active ingredient layer. The pharmacological active ingredient may be in the form of an uncharged molecule, composite or a pharmacologically acceptable salt thereof. The pharmacologically active ingredient also includes derivatives of the active ingredients (for example, ether, ester, amide, etc.) which can be readily hydrolyzed by pH in the body or enzyme, etc.

For the pharmacologically active ingredient useful in the present invention, antidepressant, as well as antihypertensive drug, antibiotics, sedative, nonsteroidal anti-inflammatory, hyperlipidemia drug, expectorant, steroids, antihistamine, analgesics, antiarthritic, febricide, immunosuppressant, protein, peptide, antitumor agent, antineoplastic, tranquilizer, hormone, somnifacient, anesthetic, antidiabetic drug, antiparkinsonian, antihemicranin and other drugs may be used. Water-soluble drugs can also be used. Preferably, the pharmacologically active ingredient is at least one material selected from the group consisting of venlafaxine, prazosin, doxazosin HCl, methylphenidate, verapamil, oxybutynin chloride, isradipine, glipizide, pseudoephedrine HCl, albuterol sulfate, bupropion, and paroxetine. Hydrochloride and derivatives of said active ingredients can also be used.

The active ingredient layer (B) comprises the pharmacologically active ingredient and may include a pharmacologically acceptable carrier, if necessary. Examples of the carrier includes an excipient, a lubricant, a pigment, a sweetening agent, etc. For the excipient, lactose or corn starch can be used. The lubricant may be at least one material selected from the group consisting of talc, magnesium stearate, magnesium silicate, calcium stearate, glyceryl behenate, polyethylene glycol, mineral oil, silicon dioxide and stearic acid. The carrier may be used in an amount of 0 to 80 wt %, as required.

The pharmacologically active ingredient is comprised in 15 to 40 wt % of the total weight of the composition for forming the active ingredient layer. If the content is less than 15 wt %, the concentration of the active ingredient is low so the amount of the granule filled in a capsule increases. If it exceeds 40 wt %, the concentration of the ingredient is high so it is difficult to control the drug release rate.

The active ingredient layer (B) also contains the water insoluble polymer. The water insoluble polymer acts as a binder which enables the pharmacologically active ingredient and the carrier to bind to the crystalline core material.

The water insoluble polymer may be at least one material selected from the group consisting of ethyl cellulose (EC), cellulose acetate (CA), polymethylmethacrylate copolymer, polyvinyl acetate and acetate-polyvinyl pyrrolidone copolymer. The water insoluble polymer is comprised in 1 to 40 wt % of the total weight of solids of the composition for forming the active ingredient layer. If the content is greater than 40 wt %, the coating solution becomes so viscous that it is not easily transferred and drying is not incompletely achieved so that the granules may aggregate.

The polymethylmethacrylate copolymer may be selected from the group consisting of commercially available Eudragit RS100, RS PO, RS 30D, RL100, RL PO, RL 30D, NE30D, and NE40D. For the vinyl acetate-polyvinyl pyrrolidone copolymer, commercially available Kollidon SR and Kollicoat SR may be used.

The active ingredient layer (B) may further comprise a water soluble polymer to facilitate coating and to increase the coating yield. The water soluble polymer may be at least one polymer selected from the group consisting of hydroxypropyl methyl cellulose (HPMC), hydroxypropylcellulose (HPC), hydroxypropyl methyl cellulose phthalate (HPMCP), polypropylene glycol, polyethylene sorbitan ester, polyethylene oxide, polyvinyl pyrrolidone, polysaccharide and poloxamer (Pluronic F68, F127).

The sustained release layer (C) is formed on a surface of the active ingredient layer (B) and comprises a sustained release film forming material so that a drug can be slowly released. The sustained release layer (C) may further comprise a plasticizer, a disintegrant, or a lubricant, if necessary.

The sustained release film forming material may be any material which can be used in the preparation of sustained release formulations, and preferably is at least one material selected from the group consisting of cellulose acetate, polymethylmethacrylate copolymer polyvinyl acetate. For the polymethyl methacrylate copolymer, commercially available Eudragit RS100, RL100, NE30D and NE40D may be used. For the polyvinyl acetate, commercially available Kollicoat SR30D and Kollidon SR may be used. The sustained release film forming material may be comprised in 1 to 25 wt % of the total weight of the sustained release drug delivery system. If the content does not lie in the range, sustained release drug delivery is difficult. Preferably, as the amount of Eudragit RS increases, release of drug delays. Likewise, for the Eudragit NE30D and Kollicoat SR30D, as the amount of the sustained release film forming material increases, dissolution of drug decreases. For Eudragit RS, Eudragit NE30D and Kollicoat SR30D, sustained release is most distinct when the amount of the film forming material is comprised in 15 to 20 wt % of the total weight of the sustained release drug delivery system. Eudragit RL exhibits the release rate of 90% or more for 1 hour in a dissolution solution.

For the plasticizer, at least one material selected from the group consisting of triethyl citrate, dibutyl sebacate, dibutyl phthalate, acetyl triethyl citrate, triacetine, glycerol, propylene glycol and polyethylene glycol having an average molecular weight of 200 to 8,500 may be used. The plasticizer may be comprised in 1 to 10 wt % of the total weight of a composition for forming the sustained release layer. If the content does not lie in the range, it is difficult to form the sustained release film.

The disintegrant is used to disperse a drug and may be a pharmaceutically acceptable disintegrant which is commonly used in the art. For example, lactose, starch, mannitol, microcrystalline cellulose, hydroxypropyl cellulose, hydroxypropyl methyl cellulose, polyprastone, etc. may be used.

The lubricant prevents granules from agglomerating, smoothens surface and gives gloss. For the lubricant, at least one material selected from the group consisting of commonly used talc, magnesium stearate, magnesium silicate, calcium stearate, glyceryl behenate, polyethylene glycol, mineral oil, silicon dioxide and stearic acid may be used. The lubricant may be comprised in 1 to 50 wt % of the total weight of the drug delivery system. If the content does not lie in the range, the coating yield decreases and the amount of granules filled in a capsule increases.

Compositions for the active ingredient layer and the sustained release layer may comprise at least one solvent such as ethanol, acetone, methylene chloride and water to perform a coating process.

The sustained release drug delivery system of the present invention is prepared in the form of granule or pellet and the plasticizer and the disintegrant are released from the film in an aqueous solution or a body fluid (gastric juice, intestinal juice, etc.) while a drug is released in a sustained release manner.

A method of preparing the sustained release drug delivery system of the present invention will now be described.

A crystalline core material is added to a coater and is coated with a composition for forming an acitive ingredient layer containing a pharmacologically active material to form an active ingredient layer on the crystalline core material. According to the coating process, the crystalline core material is spherical. Then, a composition for forming a sustained release layer containing a sustained release film forming material is coated on the active ingredient layer to form a sustained release layer on the active ingredient layer. For the coater, a fluidized bed coater is generally used, but various coaters can be used as required. The condition for the fluidized bed coater for preparing each coating layer is not particularly restricted.

### BRIEF DESCRIPTION OF THE DRAWINGS

FIG. 1 is a graph illustrating the dissolution rate of venlafaxine with time of the sustained release drug delivery system prepared in Example 1 and the commercially available EFEXOR *XR of Comparative Example 1.

FIG. 2 is a graph illustrating the dissolution rate of venlafaxine with time of the sustained release drug delivery system prepared in Example 2 and the commercially available EFEXOR *XR of Comparative Example 1.

### BEST MODE FOR CARRYING OUT THE INVENTION

Practical and presently preferred embodiments of the present invention are illustrative as shown in the following Examples and Comparative Examples.

However, it will be appreciated that those skilled in the art, on consideration of this disclosure, may make modifications and improvements within the spirit and scope of the present invention.

### Example 1

1) Coating of Active Ingredient Layer

4.71 wt % of sugar (pellet size: 600-710 μm) was added to a fluidized bed coater and was coated with a composition for forming an active ingredient layer. The composition for forming an active ingredient layer was prepared by dissolving and dispersing 4.71 wt % of corn starch, 4.0 wt % of venlafaxine hydrochloride, 1.41 wt % of Eudragit RS, 0.14 wt % of triethyl citrate, 1.7 wt % of talc, 0.94 wt % of magnesium stearate, 47.08 wt % of dichloromethane, and 35.31 wt % of ethanol. The coating condition was: inlet air pressure=5.8 bar, spray air pressure=1.2 bar, outlet air temperature=28° C., inlet air temperature=30° C., flow rate=12, and outlet air flat=30%.

2) Coating of Sustained Release Layer

47.62 wt % of the coated pellet (850-1000 μm) was coated with a composition for forming a sustained release layer. The composition for forming a sustained release layer was prepared by mixing 9.52 wt % of Eudragit NE30D, 33.34 wt % of purified water, and 9.52 wt % of talc. The coating condition was: inlet air pressure=5.8 bar, spray air pressure=0.6 bar, outlet air temperature=28° C., inlet air temperature=29° C., flow rate=6, and outlet air flat=30%.

### Example 2

1) Coating of Active Ingredient Layer

4.13 wt % of sugar (pellet size: 600-710 μm) was added to a fluidized bed coater and was coated with a composition for forming an active ingredient layer. The composition for forming an active ingredient layer was prepared by dissolving and dispersing 4.95 wt % of venlafaxine hydrochloride, 1.32 wt % of Eudragit RS, 3.30 wt % of cellulose acetate, 1.03 wt % of hydroxypropyl methyl cellulose, 0.46 wt % of dibutyl cebacate, 1.11 wt % of talc, 1.11 wt % of magnesium stearate, 20.65 wt % of acetone, 30.97 wt % of ethanol and 30.97 wt % of dimethyl chloride. The coating condition was: inlet air pressure=5.8 bar, spray air pressure=1.2 bar, outlet air temperature=28° C., inlet air temperature=30° C., flow rate=12, and outlet air flat=30%.

2) Coating of Sustained Release Layer

13.12 wt % of the coated pellet (850-1000 μm) was coated with a composition for forming a sustained release layer. The composition for forming a sustained release layer was prepared by dissolving or dispersing 2.62 wt % of Eudragit RS100, 0.26 wt % of dibutyl cebacate, 1.05 wt % of talc, 0.26 wt % of magnesium stearate, 39.37 wt % of acetone, 39.37 wt % of ethanol, and 3.95 wt % of purified water. The coating condition was: inlet air pressure=5.8 bar, spray air pressure=0.6 bar, outlet air temperature=28° C., inlet air temperature=30° C., flow rate=6, and outlet air flat=30%.

### Comparative Example 1

In this Example, EFEXOR *XR (Wyeth) containing venlafaxine hydrochloride as a commercially available antidepressant was used. The EFEXOR *XR was a capsule prepared by mixing venlafaxine hydrochloride with ethyl cellulose, hydroxypropyl methyl cellulose, titanium oxide and iron oxide and comprised 75 mg of venlafaxine per capsule.

For the EFEXOR *XR, dissolution was performed with 900 ml of purified water according to USP method I (Basket, 100 rpm). The dissolution rate was: 30% for 2 hr, 30-55% for 4 hr, 55-80% for 8 hr, 65-90% for 12 hr and 80% or more for 24 hr.

### Experimental Example 1

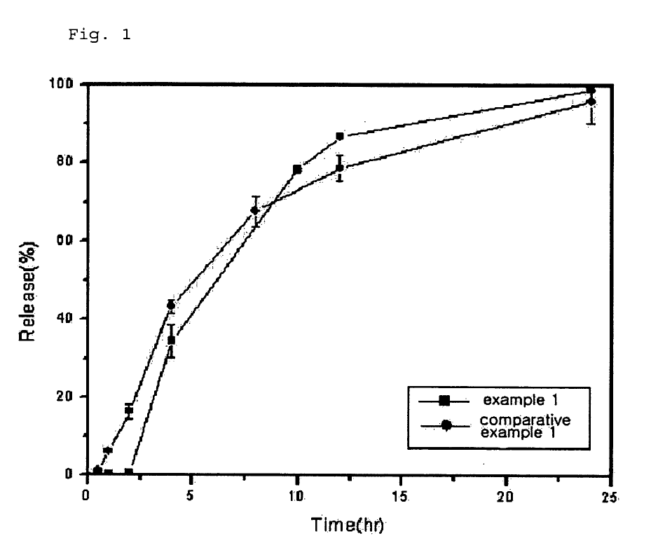

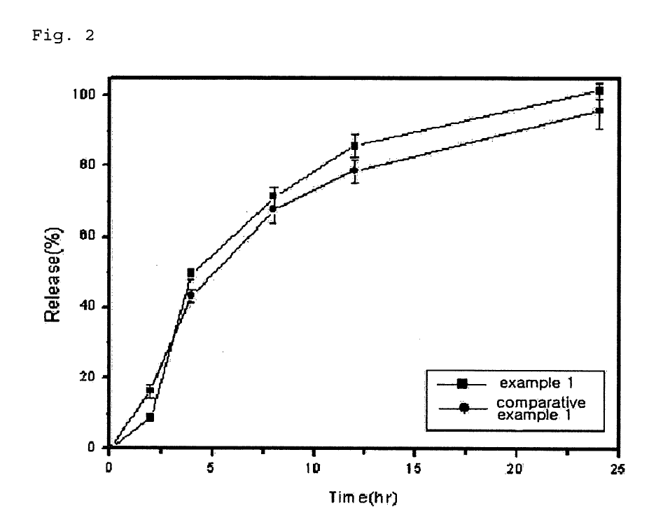

The dissolved amount of the venlafaxine hydrochloride containing granules prepared according to Examples 1 and 2 and venlafaxine of EFEXOR *XR of Comparative Example 1 was analyzed with HPLC under the following conditions. The dissolution rate is shown in Table 1. FIGS. 1 and 2 are graphs illustrating the dissolution rate with time.

Mobile phase: acetonitrile/0.02M phosphate buffer (pH 3.8)=3/7 by volume

Column: SC-04 (125*4.0 mm), PRONTOSIL EUROBOND C18 5.0 μm

Flow rate: 1.0 mL/min

Detector: 229 nm

Dissolution condition: 37.5° C., 900 ml of purified water

TABLE 1

| | | Dissolution rate (%) | | |
|---|---|---|---|---|
| | | | | Comparative |
| | | Example 1 | Example 2 | Example 1 |
| Time | 2 hr | 1 | 9 | 16 |
| | 4 hr | 34 | 40 | 39 |
| | 8 hr | 70 | 72 | 66 |
| | 12 hr | 86 | 83 | 78 |
| | 24 hr | 98 | 97 | 94 |

As can be seen from Table 1, the sustained release drug delivery systems of Examples 1 and 2 slowly release a drug at a consistent rate for 24 hours, and thus they are very suitable sustained release preparations.

### INDUSTRIAL APPLICABILITY

As described above, the sustained release drug delivery system according to the present invention can be simply prepared in a two-staged process by using the pharmacologically active ingredient and the water insoluble polymer together in the active ingredient layer and mass production thereof is possible. In addition, the release rate of a pharmacologically active ingredient can be effectively controlled by varying the type of the water insoluble polymer, the thickness of the active ingredient layer, the type of a sustained release film forming material and the thickness of the sustained release layer.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A sustained release drug delivery system comprising:
a crystalline core material (A);
an active ingredient layer (B) which is formed on an outer surface of the crystalline core material (A) and comprises 15-40 wt% of venlafaxine or its pharmacologically acceptable salt as the active ingredient and 1-45 wt% of a water insoluble polymer selected from the group consisting of polymethylmethacrylate copolymer, cellulose acetate and a mixture thereof, the weight percentages being based on the total weight of a composition for forming the active ingredient layer; and
a sustained release layer (C) which is formed on an outer surface of the active ingredient layer (B) and comprises 1-25 wt% of polymethylmethacrylate copolymer based on the total weight of the sustained release drug delivery system, as a sustained ieleaee film forming material.

2. The sustained release drug delivery system of claim 1, wherein the crystalline core material (A) is a granular material selected from the group consisting of sugar, sugar granule, nonpareil, and sugarsphere.

3. The sustained release drug delivery system of claim 2, wherein the crystalline core material (A) is a pellet having a particle size of 300 to 900 μm.

4.-7. (canceled)

8. The sustained release drug delivery system of claim 1, wherein the active ingredient layer (B) further comprises a water soluble polymer.

9. The sustained release drug delivery system of claim 8, wherein the water soluble polymer is at least one material selected from the group consisting of hydroxypropyl methyl cellulose (HPMC), hydroxypropyl cellulose (HPC), hydroxypropyl methyl cellulose phthalate (HPMCP), polypropylene glycol, polyethylene sorbitan ester, polyethylene oxide, polyvinyl pyrrolidone, polysaccharide and poloxamer.

10.-11. (canceled)

12. The sustained release drug delivery system of claim 1, wherein the active ingredient layer (B) and the sustained release layer (C) further comprise at least one additive selected from the group consisting of an excipient, a disintegrant, a lubricant and a plasticizer.

13. The sustained release drug delivery system of claim 12, wherein the excipient is lactose or corn starch and the lubricant is at least one material selected from the group consisting of talc, magnesium stearate, magnesium silicate, calcium stearate, glyceryl behenate, polyethylene glycol, mineral oil, silicon dioxide and stearic acid.

14. The sustained release drug delivery system of claim 12, wherein the plasticizer is at least one material selected from the group consisting of triethyl citrate, dibutyl sebacate, dibutyl phthalate, acetyl triethyl citrate, triacetine, glycerol, propylene glycol and polyethylene glycol having an average molecular weight of 200 to 8,500.

15. The sustained release drug delivery system of claim 12, wherein the plasticizer is comprised in 1 to 10 wt % of the total weight of the sustained release drug delivery system.

16. The sustained release drug delivery system of claim 12, wherein the lubricant is comprised in 1 to 20 wt % of the total weight of the sustained release drug delivery system.

17. The sustained release drug delivery system of claim 1, wherein the active ingredient layer (B) and the sustained release layer (C) are formed using a fluidized bed coater.
